(19) United States
(12) Patent Application Publication
(10) Pub. No.: US 2007/0282342 A1
(43) Pub. Date: Dec. 6, 2007

(54) Articulated Bone Screw

(76) Inventors: Alfred Niederberger, Grenchen (CH); Christian Van Der Werken, Zeist (NL)

Correspondence Address: JONES DAY, 222 EAST 41ST STREET, NEW YORK, NY, 10017-6702, US

(21) Appl. No.: 10/593,600

(22) PCT Filed: Mar. 26, 2004
(86) PCT No.: PCT/CH04/00187

§ 371 Date: May 16, 2007

(51) Int. Cl. A61B 17/58

(52) U.S. Cl. 606/73; 606/60; 606/72

(57) ABSTRACT

The bone screw has a head, a shaft and a longitudinal axis. The shaft comprises a proximal section adjacent to the head and a distal section secured to the proximal section by a cardan or cardan-like joint, the distal section being suitable for introduction into the bone and being provided with an outer thread. The bone screw can be bent in all directions relative to its longitudinal axis, while retaining its axial rigidity and fully transmitting a couple without the need for additional means.

### FIELD OF THE INVENTION

The invention relates to a bone screw.

### BACKGROUND OF THE INVENTION

U.S. Pat. No. 4,959,064 of ENGLEHARDT discloses a bone screw with a spring part. The spring part of the bone screw endows it with a certain axial elasticity (axial compression or distraction), as well as a certain torsion and also a certain radial bending in all direction. Accordingly, this known bone screw will prevent only a decrease in the compressive effect of the screw.

The EP-A 1,273,269 of MÜCKTER discloses a bone screw with an elastic shaft. The elasticity of this construction as described in various embodiments. Here also, the elastic connection of the screw provides a certain axial elasticity. However, the transfer of torques is not possible without the help of further stabilizing instruments or implants.

### SUMMARY OF THE INVENTION

It is an object of the invention to create a bone screw, which can be bent in all directions relative to its longitudinal axis while, at the same time, it retains its axial rigidity and transmits a torque fully without the help of additional means.

The advantages, achieved by the invention, can be seen to lie essentially therein that, due to the inventive bone screw, bones can be connected with one another in such a manner that a certain bending and, with that, mobility with respect to one another becomes possible.

In the case of a special embodiment, the cardan joint consists of a classical universal joint. In the case of a different embodiment, the cardan-like joint consists of a ball joint with a ball head, which has a polygonal, preferably octagonal cross-section and a ball socket, which is suitable for accommodating the cross section of the ball head. This construction with a spherical octagon has the advantage that the design of the construction is simplified, while the freedom is somewhat limited in comparison to the classical universal joint.

In the case of a further embodiment, the bone screw has several universal or cardan-like joints. The greater degree of freedom of the bone screw is an advantage.

In the case of a further embodiment, the bone screw has a continuous cannulation, which extends coaxially with its longitudinal axis. This permits the cardan joint to be blocked by introducing a Kirschner wire into the cannulation.

Advantageously, the length of the shaft, which is formed from the sum of the two lengths of the proximal section and the distal section, is constant.

In the case of a preferred embodiment, the proximal section also is provided at least partially with an external thread. If a certain distance is to be maintained between the bone parts, the latter can be drilled out with a suitable drill, so that the thread in the distal section as well as that in the proximal section then engage. The distance between the two bone parts now remains adjusted fixed at a particular value. In this way, the bone screw is used as a setting screw.

On the other hand, if a change in the distance between the two bone parts is to be permitted, the borehole, which is intended to accommodate the proximal part of the screw, is drilled with a diameter larger than the external thread of the proximal part. The proximal thread now does not engage the proximal bone part (for example, in the collarbone). The distance between the bone parts can now be changed. Of course, this can be done only within certain limits, since the maximum distance is limited by the screw head. Accordingly, in this case, the bone screw is used as a tension screw.

Accordingly, the bone screw with the proximal thread can be used more universally. If, for example, the bone parts are to consolidate together, the bone screw is used as a tension screw and is screwed in up to the coracoid process.

In the case of a special embodiment, the bone screw is constructed to be self-cutting and/or self-drilling.

For a further embodiment, the deflection of the joint is limited preferably to not more than 90° and advisably to not more than 30°.

### BRIEF DESCRIPTION OF THE DRAWINGS

The invention and further developments of the invention are explained in even greater detail in the following by means of the partly diagrammatic representations of several examples, of which:

FIG. 1 shows a perspective view of the bone screw with a cardan joint and with a Kirschner wire that has been introduced,

FIG. 2 shows a longitudinal section through the bone screw in the linearly aligned state of the Kirschner wire,

FIG. 3 shows a perspective view of the bone screw of FIG. 2 in the bent state without a Kirschner wire,

FIG. 4 shows a partial longitudinal section through a modified cardan-like ball joint for a bone screw,

FIG. 5 shows a view of a bone screw of FIGS. 1-3, implanted in the shoulder region, and

FIG. 6 shows a view of the bone screw of FIGS. 1-3, implanted in the region of the ankle joint.

### DETAILED DESCRIPTION OF THE PREFERRED EMBODIMENTS

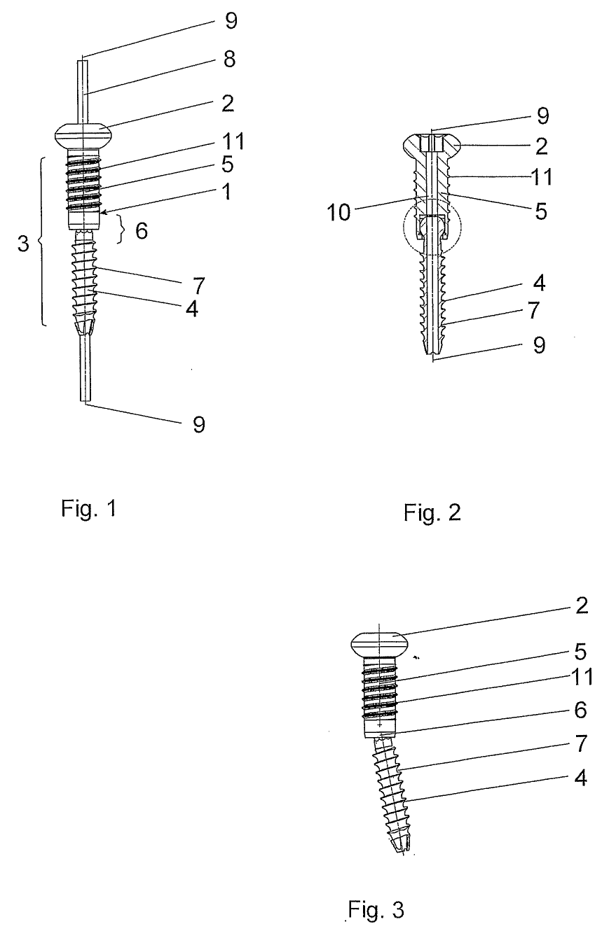

The inventive bone screw 1, shown in FIGS. 1-3, has a head part 2, a shaft 3, a longitudinal axis 9 and a continuous central cannulation 10. The shaft 3 consists of a proximal section 5 with an external thread 11, adjoining the head part 2, and a distal section 7 with an external thread 4, fastened thereto by means of a cardan joint 6, for introduction into the bone. If a Kirschner wire 8 (FIG. 1) is introduced into the central cannulation 10 (FIG. 2), the cardan joint 6 is blocked, so that the bone screw 1 can no longer be bent, as indicated in FIG. 1.

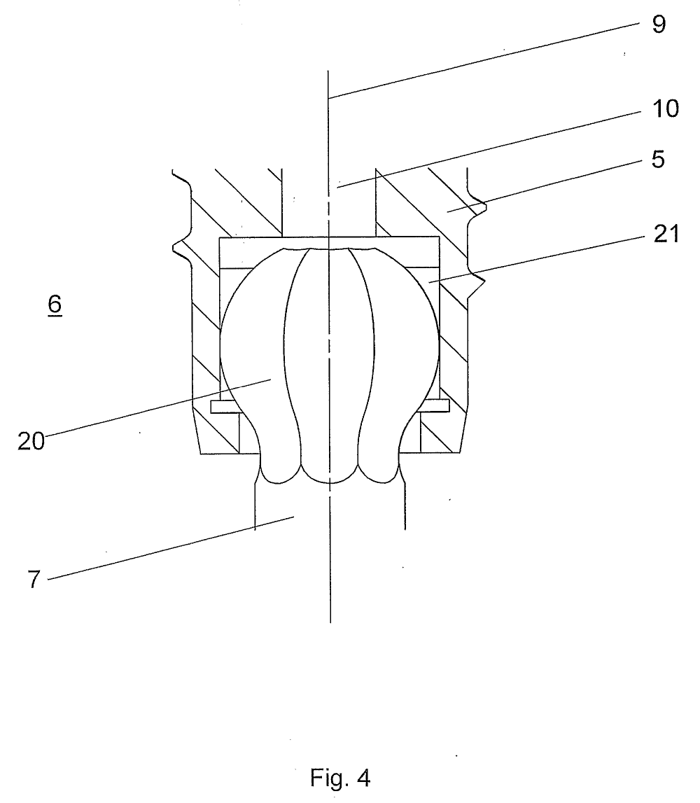

As shown in detail in FIG. 4, the cardan joint 6 consists of a ball joint with a ball head 20 and a ball socket, which is accommodated in the proximal section 5. The ball head 20 has an octagonal cross section and the ball socket has a correspondingly adapted, octagonal geometry, which is suitable for accommodating the cross section of the ball head 20. This geometry permits the bone screw 1 to be rotated also in the bent state. Instead of a cardan joint, it is also possible to use a classical universal joint.

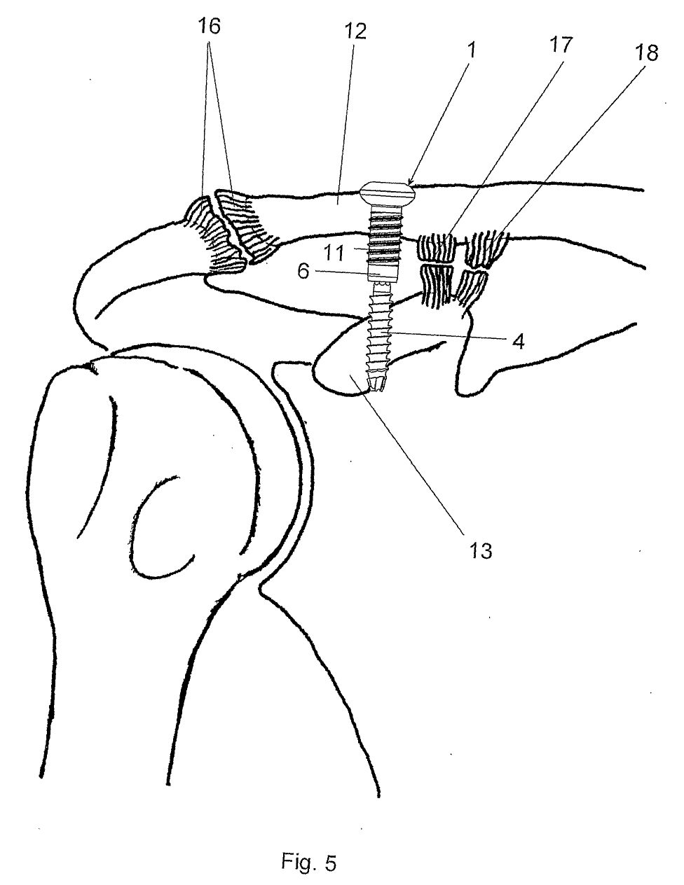

FIG. 5 shows a use of the inventive bone screw 1 in the shoulder region for producing a connection between the collarbone 12 and the coracoid process 13. Normally, the collarbone 12 is held in position by the Lig. acromiclaviculare 16, Lig. trapezoidum 17 and the Lig. conoideum 18. If these ligaments are ruptured, the collarbone 12 is no longer held in position and the so-called “piano keys effect” occurs. At the same time, the collarbone 12 is pulled upward by the muscles and then protrudes in the region of the shoulder joint. The surgical treatment according to the prior art consists therein that the ligaments are sewn together and a rigid bone screw is introduced for a certain time (several months), so that forces do not act on the ligaments. Unfortunately, rigid bone screws break under the given conditions. The use of the inventive bone screw 1 (instead of a rigid screw) permits the collarbone 12 and the coracoid process 13, which have become movable with respect to one another, to be connected so that the ligaments 16, 17, 18, which have been sewn together, remain stress free. Bending of the two bones is permitted by the cardan joint 6 of the bone screw 1; at the same time, the distance between the two bones is retained

The surgical technique for this use in the shoulder region is described briefly below:

a) the Kirschner wire 8 is threaded through the collarbone 12 into the coracoid process 13,

b) a hole through the collarbone 12 is drilled by way of the Kirschner wire 8 by means of a pierced drill. The coracoideus process 13, on the other hand, is not pre-drilled;

c) the bone screw 1 is introduced over the Kirschner wire 8 into the hole pre-drilled in the collarbone 12 and screwed with its external thread 4 in to the coracoid process 13. The Kirschner wire 8 stabilizes the bone screw 1 during this time and also guides it and

d) the Kirschner wire 8 is removed and, as a result, the bone screw 1 can be bent.

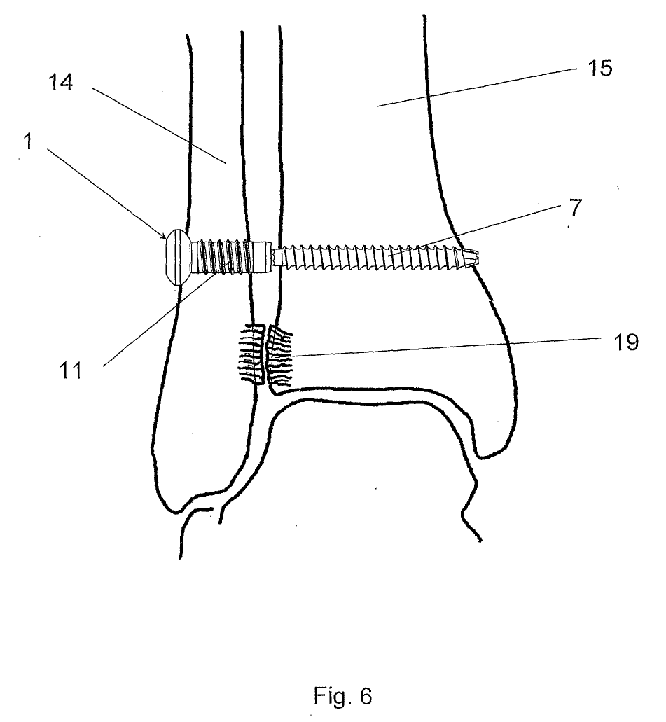

FIG. 6 shows a first application of the inventive bone screw 1 in the region of the ankle joint, in order to produce a connection between the fibula 14 and the tibia 15. The ligament 19 (syndesmosis) is torn so that the fibula 14 and tibia 14 drift apart. The ligament 19 is sutured pursuant to the prior art. By using the inventive bone screw 1, the stress on the sutured ligament 19 can be relieved while the distance between the two bones is maintained. The surgical technique employed is similar to that, which was described in connection with FIG. 5.

1-10. (canceled)

11. A bone screw comprising:
a head,
a shaft having a longitudinal axis, a proximal section adjoining the head, and a distal section;
wherein the distal section is attached to the proximal section by way of a ball-and-socket joint.

12. The bone screw of claim 11, wherein the distal section comprises the ball, and the proximal section comprises the socket.

13. The bone screw of claim 11, wherein the proximal section is at least partially threaded.

14. The bone screw of claim 11, wherein the distal section is at least partially threaded.

15. The bone screw of claim 11, wherein the shaft is cannulated.

16. The bone screw of claim 11, wherein the length of the shaft is constant.

17. The bone screw of claim 11, wherein the proximal section has a larger diameter than the distal section.

18. The bone screw of claim 11, wherein the distal section is allowed to deflect no more than about 90 degrees relative to the longitudinal axis.

19. The bone screw of claim 11, wherein the distal section is allowed to deflect no more than about 30 degrees relative to the longitudinal axis.

20. The bone screw of claim 11, wherein the ball has a substantially octagonal shape.

21. The bone screw of claim 11, wherein the distal section is rotatable relative to the proximal section about the longitudinal axis.

22. A method for inserting a bone screw comprising:
inserting a guide wire into the body;
drilling a first hole into a first bone segment;
inserting the bone screw over the guide wire and at least partially within the first hole; and
removing the guide wire, thereby allowing the screw to bend.

23. The method of claim 22, wherein the bone screw has a head, and a shaft having a longitudinal axis, a proximal section adjoining the head, and a distal section; wherein the distal section is rotatably attached to the proximal section.

24. The method of claim 22, wherein the bone screw has a cannulated shaft.

25. The method of claim 22, wherein the first bone segment is a collarbone.

26. The method of claim 22, further comprising the step of inserting at least a portion of the bone screw into a second bone segment.

27. The method of claim 26, wherein the second bone segment does not have a pre-drilled hole.

28. The method of claim 26, wherein the second bone segment is a coracoideus process.

29. The method of claim 26, wherein the bone screw has a shaft having a proximal section inserted into the first bone segment and a distal section inserted into the second bone segment.

30. A bone screw comprising:
a head,
a shaft having a longitudinal axis, a proximal section adjoining the head, and a distal section polyaxially associated with the proximal section.
